(19) United States
(12) Patent Application Publication
(10) Pub. No.: US 2019/0308018 A1
(43) Pub. Date: Oct. 10, 2019

(54) DETERMINING EYE MOVEMENT USING INDUCTION

(71) Applicant: THE BIONICS INSTITUTE OF AUSTRALIA, East Melbourne, Victoria (AU)

(72) Inventors: Matthew Alexander Petoe, East Melbourne, Victoria (AU); Christopher Edward Williams, East Melbourne, Victoria (AU); Rodney Millard, East Melbourne, Victoria (AU); Joel Villalobos, East Melbourne, Victoria (AU); Peter Misha Seligman, East Melbourne, Victoria (AU)

(21) Appl. No.: 16/307,847

(22) PCT Filed: Jun. 6, 2017
(86) PCT No.: PCT/AU2017/050557

§ 371 Date: Dec. 6, 2018

(30) Foreign Application Priority Data
Jun. 7, 2016 (AU) 2016902205

(51) Int. Cl. A61N 1/36; A61N 1/05

(52) U.S. Cl. CPC A61N 1/36046; A61N 1/025; A61N 1/0543

(57) ABSTRACT

The present disclosure relates to visual prosthesis apparatus including an implantable device having a substrate and a plurality of electrodes located in or on the substrate, the substrate adapted to be implanted at least partially in an eye of a patient. A first inductor is included in the implantable device, for example by encapsulating an inductor coil in the substrate or on an associated lead or anchor device. In some instances, the electrodes in the substrate may partially provide the first inductor. A second inductor is adapted to locate externally to the eye and inductively couple with the first inductor. A processor is adapted to determine a direction of movement of the eye based on changes in electrical current induced in one of the first and second inductors due to relative movement of the first and second inductors.

### CROSS-REFERENCE TO RELATED APPLICATIONS

The present application claims priority to Australian Provisional Application No. 2016902205 filed on 7 Jun. 2016, the content of which is herein incorporated by reference.

### TECHNICAL FIELD

The present disclosure relates to apparatus for stimulating neurons of a patient to provide a visual percept to the patient's brain.

### BACKGROUND

Visual prostheses have been developed to stimulate neurons to provide a visual percept to a blind or partially blind patient. A visual prosthesis commonly includes an implantable device having an electrode array for placement in the eye on or near retinal nerve cells. Alternatively, the electrodes may be placed directly on the visual cortex of the brain. Electrical signals are transmitted via the electrodes to the retinal nerve cells or visual cells in the brain, triggering a perception of vision within the patient's brain. The prosthesis can therefore restore or improve vision perception to patients whose retinal photoreceptors have become dysfunctional, for example.

Commonly, a visual prosthesis is used in conjunction with an image capture device such as a video camera. Based on a series of images detected by the camera, electrical signals are delivered to the electrode array to stimulate nerve cells to create a visual percept.

Tracking of the position and/or movement of the eye enables spatially accurate information to be provided to a patient using a visual prosthesis. When a direction of eye gaze of the patient changes, tracking of the eye enables an analogous change in direction of the visual percept to be elicited. Eye tracking can be performed using a video camera trained on the eye. However, image processing of a video signal from the video camera, to determine changes in direction of gaze, can be time consuming and can introduce significant time delays in the production of the visual percept, which time delays can impair navigation ability and orientation perception of the patient.

Scleral search coils have also been used to track eye movement. A coil is embedded into a contact lens that sits on the patient's eye. Alternating magnetic fields are generated around the eye, inducing current in the coil by virtue of electromagnetic induction. Changes in the current occur as the coil moves within the magnetic fields, and these current changes can be monitored to determine eye movement.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

According to an aspect of the present disclosure there is provided visual prosthesis apparatus comprising:

an implantable device comprising a substrate and a plurality of electrodes located in or on the substrate, the substrate adapted to be implanted at least partially in an eye of a patient;

a first inductor comprised in the implantable device; and

a second inductor adapted to locate externally to the eye and inductively couple with the first inductor; and

a processor adapted to determine a direction of movement of the eye based on changes in electrical current induced in one of the first and second inductors due to relative movement of the first and second inductors.

The apparatus may comprise an image capture device adapted to locate externally to the eye and capture one or more images. The processor may provide a stimulation signal to the plurality of electrodes based on the one or more captured images. The processor may directly adapt the stimulation signal based on the determined direction of movement of the eye. Additionally or alternatively, the processor may direct movement of the image capture device based on the determined direction of movement of the eye.

The adaptation of the stimulation signal and/or directing of movement of the image capture device, based on the determined direction of movement of the eye, may be performed to give the patient the visual percept of scanning across a visual scene in accordance with their eye movement, and/or the visual percept of maintaining fixation on the same portion of a visual scene as the patient's head moves.

Additionally or alternatively, the adaptation of the stimulation signal and/or directing of movement of the image capture device, based on the determined direction of movement of the eye, may be used to counteract effects of involuntary eye movement. Examples of involuntary eye movement include nystagmus. In general, eye movement can change the perceived position of a visual percept by a patient's brain, and this can be particularly problematic in the case of involuntary eye movement as it can lead to undesirable changes in the perceived position of the visual percept. The adaptation of the stimulation signal, and/or the directing of movement of the image capture device, may therefore be based at least partially on determined involuntary eye movement.

In addition or as an alternative to controlling the delivery of the stimulation signal, the monitoring of eye movement may be used to provide feedback on eye movement, e.g. if the eye movement exceeds desirable limits. For example, the processor may determine if movement of the eye has exceeded a threshold level and may issue an alert signal if the threshold level is exceeded. The alert may be an auditory or haptic alert, for example. The threshold level may relate to a rate of eye movement or a position of the eye movement. The feedback may assist the patient in appropriately viewing a visual scene. The feedback may ensure that the patient does not move their eye beyond the limits in which compensation techniques as described above are capable of working.

The apparatus may comprise a current source that supplies an alternating current to one of the first and second inductors, resulting in an alternating magnetic field being produced around that inductor and current being induced in the other of the first and second inductors due to the inductive coupling. Relative movement of the first and second inductors causes changes to the induced current (e.g. changes in polarity, phase, and/or amplitude). The apparatus may comprise a sensor, e.g. a sensitive ammeter or voltmeter, to monitor changes in the induced current. Based on the monitored changes, a degree of relative movement of the first and second inductors, e.g. along an anatomical axis, can be determined.

In another aspect of the present disclosure there is provided an implantable device comprising:

a substrate and a plurality of electrodes located in or on the substrate, the substrate adapted to be implanted at least partially in an eye of a patient;

a first inductor comprised in the implantable device; the first inductor adapted to inductively couple with a second inductor located externally to the eye to enable determination a direction of movement of the eye based on changes in electrical current induced in one of the first and second inductors due to relative movement of the first and second inductors.

In any of the aspects, the inductors may take a variety of forms. For example, the first inductor may comprise a partial turn, single turn or a plurality of turns of wire and/or the second inductor may comprise a partial turn, a single turn or a plurality of turns of wire. Where the inductor comprises a plurality of turns of wire, it may be considered a coil. Where the inductor comprises only one turn or a partial turn, it may be considered a loop or partial loop, respectively.

The first inductor may be a first coil and/or the second inductor may be a second coil. Alternatively, the first inductor may be a first loop or partial loop and/or the second inductor may be a second loop or partial loop. In one embodiment, first inductor is a first partial loop and the second conductor is a second coil. The first partial loop may comprise a first conductor connected to a first electrode of the plurality of electrodes and a second conductor connected to a second electrode of the plurality of electrodes, wherein the first and second electrodes are electrically connectable through tissue of the eye to provide a complete loop.

In any of the aspects, the first inductor may be located in or on the substrate. The first inductor may be encapsulated by the substrate. The first inductor may be located at an end of the substrate substantially opposite to an end at which the electrodes are located. For example, the electrodes may be located at or adjacent a distal end of the substrate and the first inductor may be located at or adjacent a proximal end of the substrate. Alternatively, as indicated above, the electrodes may provide, at least in part, the first inductor.

The implantable device may further comprise a lead, through which one or more conductors extend from the electrodes and substrate to a region external to the eye, to enable electrical communication with the electrodes. A proximal region of the lead may be partially implanted in the eye along with the substrate. A distal region of the lead may not be implanted in the eye. The proximal region may be at least partially implantable at the side of the patient's head. For example, the lead may extend from the eye, past extra-ocular muscles and subcutaneously around the orbital bone at the lateral orbital margin.

The implantable device may comprise an anchor device adapted to support one or more conductors and/or the lead as they extend out of an opening in an outer surface of the eye. The anchor device may be adapted to locate over the opening in the outer surface of the eye. The anchor device may be fixed to the outer surface of the sclera adjacent the opening.

When a lead is provided, as an optional alternative to locating the first inductor in or on the substrate, the first inductor may be located in or on the lead. The first inductor may be mounted on the lead using a fixation means such as a clamp or other mount, or using adhesive, stapling, suturing or otherwise. Alternatively, the first inductor may be encapsulated by the lead, e.g. within a cladding layer of the lead.

When an anchor device is provided, as an optional alternative to locating the first inductor in or on the substrate, or in or on the lead, the first inductor may be located in or on the anchor device. The first inductor may be mounted on the anchor device using a fixation means such as a clamp or other mount, or using adhesive, stapling, suturing or otherwise. Alternatively, the first inductor may be encapsulated by the anchor device, e.g. within silicone or other material that may form the anchor device.

By providing the first inductor as part of the implantable device, fewer steps may be required to deploy the apparatus. The stimulation electrodes and the first inductor can be deployed substantially at the same time. The substrate, lead and/or anchor device may be implanted using existing surgical techniques and no new or additional surgical techniques may be required in order to implant the first inductor. Further, the implantable device can provide a secure location for the first inductor within or on the eye and therefore any additional stabilisation components may not be necessary. Further, providing the first inductor in the implantable device may reduce any discomfort to the patient that might otherwise be found, e.g. through use of an additional support such as a contact lens. Still further, since the position of the substrate, anchor device, and at least a part of the lead, may remain substantially fixed in relation to the eye, reliable positioning of the first inductor relative to the eye may be achievable. This may contrast to an arrangement in which the first inductor is supported by a contact lens, which contact lens may be prone to migration over the surface of the eye during use.

The second inductor may be adapted to locate in a substantially fixed relationship with a portion of the body adjacent the eye, e.g., an eye socket of the patient. The second inductor may be comprised in eyewear such as spectacles worn by the patient. The eyewear may comprise the image capture device.

The apparatus may comprise a plurality of the second inductors adapted to be located externally to the eye, e.g., a plurality of second coils. For example, three, four or more second inductors may be provided. The second inductors may be oriented at different angles from each other, e.g. located orthogonally to each other or otherwise. Each second inductor, in combination with the first inductor, may be used to monitor movement along a different axis. For example, one of the second inductors may be used to monitor lateral eye movement, which is generally the most prevalent of eye movements. Another of the second inductors may be used to monitor vertical (superior-inferior) movement. Yet another of the second inductors may be used to monitor torsional movement.

As an alternative or in addition to providing second inductors that are located orthogonally to each other, second inductors may be provided that are angled so that the axis of each second inductor (e.g. a coil axis) points substantially towards the first inductor of the implantable device. Where three second inductors are provided, the inductors may be positioned in a triangular arrangement, e.g. so that each of the second inductors is located at the apex of a notional triangle. The notional triangle may be an equilateral triangle or otherwise. Where four second inductors are provided, the second inductors may be positioned in a square, rectangular, rhombic or trapezoidal arrangement, e.g. so that each of the second inductors is located at the corner of a notional square, rectangle, rhombus or trapezoid.

Alternating current may be applied to either one of the first and second inductors to generate an alternating magnetic field, with changes in the electrical current being induced at the other of the first and second inductors as it moves within the magnetic field.

When alternating current is supplied to multiple second inductors, the current may be supplied to each of the second inductors at a different frequency, such that an alternating magnetic field of a different frequency is generated at each second inductor. The electrical current (electrical signal) induced in the first inductor within the different magnetic fields can be demodulated on a frequency-specific basis to identify movement along each second inductor axis.

As an alternative, when alternating current is supplied to multiple second inductors, the alternating current may be supplied to each of the second inductors at different time periods, such that an alternating magnetic field is generated at each second inductor at different times. The electrical signal induced in the first inductor within the different magnetic fields can be demodulated on a time-specific basis to identify movement along each second inductor axis.

When alternating current is supplied to the first inductor, electrical signal induced in each second inductor within the magnetic field can be monitored independently to identify movement.

Additionally or alternatively, different types of current signals can be supplied to different inductors to enable differentiation of relative movement between different inductor combinations. For example, the current signals may have different shapes e.g. square wave shapes or a triangular wave shapes or otherwise.

As indicated above, multiple second inductors may be provided, which can be at different locations and can be oriented at different angles from each other, e.g. located orthogonally to each other or otherwise. In some embodiments, at any particular location, second inductors, e.g. second coils, may be employed in groups. In one embodiment, where second inductors are positioned at different locations and oriented orthogonally or at other angles to each other, at each location a group of the second inductors is provided. In another embodiment, only one group of the second inductors is provided. A group of second inductors may comprise a pair of second inductors or 3 or more second inductors.

Where one or more groups of second inductors are provided, each group may be used in differential configuration. The second inductors of a group may have the same orientation and be located in substantially the same plane as each other, but may be displaced from each other (e.g. vertically displaced, horizontally displaced or otherwise). The second inductors in the group may also be in an inversely polarised relationship. For example, the second inductors, particularly when in the form of coils, may have opposing directions of coil winding (e.g. one may be clockwise and the other anticlockwise) such that current signals received from the coils are inverted relative to each other. As an alternative, inversion between signals may be achieved by connecting the second inductors to the processor with an inverted relationship or configuring the processor to invert a signal received from one of the second inductors but not a signal received from one or more others of the second inductors. The displacement between the second inductors will cause differences in the induced current signals at each second inductor. Moreover, the inversely polarised relationship between the second inductors will cause an inverse relationship between the signals. By comparing the different signals from the second inductors of a group, e.g., through a summation of the inversed signals, direction of eye movement may be determined. In general, the use of one or more groups of second inductors in differential configuration may therefore enable eye movement in different directions to be detected by the apparatus and/or may increase the accuracy or resolution of the eye movement detected by the apparatus.

When current is supplied to the first inductor for the purpose of eye tracking, the current may be the same stimulation current that is delivered to the plurality of electrodes of the substrate for the purposes of providing a visual percept of an image to the patient, or may at least be from the same current source. Stimulation current can be sent to the plurality of electrodes, based on the one or more captured images to provide a visual percept to the patient based on the captured images, e.g. for the purposes of restoring or replicating visual function. As discussed above, however, the plurality of electrodes may also provide in part the first inductor. In this instance, stimulation current can also generate a magnetic field and therefore induce current in the one or more second inductors by virtue of electromagnetic induction. Thus, some or all of the current signals that are used to create a visual percept based on captured images may also be used for the purpose of tracking eye movement through inductive coupling. This may reduce the complexity of the apparatus, enabling a single current source to be used, for example. Nevertheless, when the plurality of electrodes provides in part the first inductor, alternative approaches may be taken. For example, current may be applied to the one or more second inductors, rather than the first inductor. A selection of the plurality of electrodes and associated conductors, e.g., that are not used for stimulation of the eye, may be used to form the first inductor.

When electrical current is applied to the first inductor of the implantable device, inducing electrical signals at multiple second inductors external to the eye, a position of the eye may be determined by comparing an electrical signal level at each of the second inductors at an instant in time or by comparing average signal levels recorded at each second inductor over a time period. Similarly, when electrical current is applied to the second inductors external to the eye, inducing separately identifiable electrical signals at the first inductor of the implantable device that are each related to a different second inductor, a position of the eye may be determined by comparing an electrical signal level for each of the separately identifiable signals at an instant in time or by comparing average signal levels over a time period. In some embodiments, the comparing of signal levels may comprise determining a ratio of the signal levels between each of the second inductors. The comparing of the signal levels may be used to determine a position of the eye and/or movement of the eye. The ratio may be referenced to a look-up table, the look-up table providing pre-determined eye position data for different ratios.

In some embodiments, electrical current may be applied to the first inductor as a series of biphasic pulses, e.g., to ensure that there is no net charge transfer to the eye. Each biphasic pulse may induce a current signal at each second inductor that comprises a plurality of positive and negative signal spikes, each signal spike corresponding to a different slope of the biphasic pulse (e.g., positive-going or negative-going voltage slopes). A detector module may be provided in the apparatus that analyses the induced signal. The detector module may process the induced signal, e.g. to provide a signal output in which any negative spike is converted into a positive spike, or vice-versa, and/or in which signal noise is filtered. In one embodiment, to filter signal noise, the detector module comprises a circuit that is gated by the biphasic signal that is being passed to the first inductor so that the induced current in the second inductor is only examined at specific times, i.e. at the known instances where changes in slope polarity at the second inductor will occur. By setting a zero baseline at the instant before or between the changes in slope polarity, a processed signal for a specific time period can be output by the detector module in which background noise is substantially eliminated.

The processed signal may be integrated over a time period (e.g. a time period corresponding to the time period during which a complete biphasic pulse is delivered to the first inductor). The detector module may summate the integrated signal over the time period to arrive at a summated signal value. Each second inductor or group of second inductors may be provided with a unique detector module or circuit, or a common detector module or circuit may be used. In general, in some embodiments, the detection and gating functions may be performed by one or more electronic circuits and in other embodiments they may be performed in part or wholly by the processor.

When electrical signals are compared, e.g. to determine ratios of signal levels as described above and/or for use with respect to a look-up table, the comparison may be based on the summated signal values.

In the present disclosure, the processor may comprise a number of control or processing modules for controlling one or more features of the apparatus and may also include one or more storage elements, for storing desired data, e.g., look-up tables, threshold levels, etc. The modules and storage elements can be implemented using one or more processing devices and one or more data storage units, which processing devices and/or storage devices may be at one location or distributed across multiple locations and interconnected by one or more communication links.

Further, the processing modules can be implemented by a computer program or program code comprising program instructions. The computer program instructions can include source code, object code, machine code or any other stored data that is operable to cause the processor to perform the steps described. The computer program can be written in any form of programming language, including compiled or interpreted languages and can be deployed in any form, including as a stand-alone program or as a module, component, subroutine or other unit suitable for use in a computing environment. The data storage device(s) may include suitable computer readable media such as volatile (e.g., RAM) and/or non-volatile (e.g., ROM, disk) memory or otherwise.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

By way of example only, embodiments are now described with reference to the accompanying drawings, in which:

FIG. 1 shows a top view of components of visual prosthesis apparatus according to an embodiment of the present disclosure;

FIGS. 2a to 2c show steps in a method of implanting apparatus of FIG. 1 according to an embodiment the present disclosure;

FIGS. 3a and 3b show example positioning of components of the apparatus of FIG. 1 relative to the corneal limbus and the optic disk, respectively;

FIGS. 4a and 4b show front and top views, respectively, of an eye socket with the apparatus of FIG. 1 positioned for use;

FIG. 5 shows positioning of a lead and anchor device of the apparatus of FIG. 1 relative to an outer surface of the eye;

FIG. 6 shows routing of the lead of FIG. 5 along the skull to a communications interface;

FIG. 7 shows eyewear employed in the apparatus of FIG. 1;

FIG. 8 shows a schematic drawing of components of the apparatus of FIG. 1;

FIGS. 9a and 9b provide illustrations of how different portions of images captured by a video camera can be selected in consideration of determined movement of the eye;

FIG. 10 shows a top view of components of visual prosthesis apparatus according to another embodiment of the present disclosure;

FIG. 11 shows a top view of components of visual prosthesis apparatus according to yet another embodiment of the present disclosure;

FIGS. 12a and 12b show top views of visual prosthesis apparatus according to another embodiment of the present disclosure;

FIG. 13 shows a schematic view of a pair of coils in a differential configuration, usable in embodiments of the present disclosure;

FIGS. 14a and 14b show a front view and an upper view, respectively, of visual prosthesis apparatus according to another embodiment of the present disclosure;

FIGS. 15a and 15b show a front view and an upper view, respectively, of visual prosthesis apparatus according to yet another embodiment of the present disclosure;

FIG. 16 shows a schematic drawing of components of the visual prosthesis apparatus of FIGS. 14a and 14b;

FIGS. 17a and 17b shows graphs of induced voltage signal, according to an embodiment of the present disclosure, caused by a magnetic field generated by one or more biphasic voltage inputs.

### DESCRIPTION OF EMBODIMENTS

Certain embodiments of the present disclosure relate to visual prosthesis apparatus that employs conductors configured to extend through a lead from a component implanted in an eye to electronics, such as a stimulation and/or monitoring device or a communications interface, remote from the eye. The interface may be a plug pedestal or other type of connector, and may comprise a wireless transmitter/receiver or comprise electrical connections for wired communication. The interface may therefore provide for wired or wireless connection between the implanted device and additional electrical components of the visual prosthesis apparatus, which additional components may be implantable or otherwise. The electronics or interface may be attached to, or wholly or partially implanted in, the side of the patient's head (or other part of the patient's anatomy). Nevertheless, alternative embodiments may not employ a lead. Communication between the implanted components and external apparatus may be carried out wirelessly.

Throughout this specification the term “visual prosthesis apparatus” is used to denote apparatus for improving a patient's vision (or at least giving improved “perception” of vision), and will be understood to include devices otherwise known as bionic eyes, artificial eyes, retinal prostheses and retinal stimulators or similar.

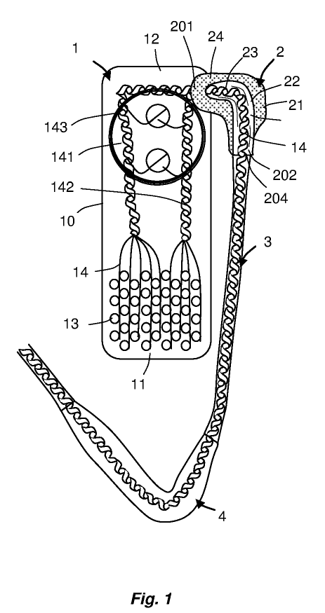

FIG. 1 shows a top view of visual prosthesis apparatus according to an embodiment of the present disclosure, the apparatus including an implantable device 1 including a substrate 10, an anchor device 2 and a lead 3.

In particular, the implantable device 1 has a flexible substrate 10 with a distal end 11 and a proximal end 12. The substrate 10, when viewed from above, is substantially rectangular, with curved corners to minimise surgical trauma, its longitudinal direction extending between the distal and proximal ends 11, 12. Adjacent the distal end 11 of the substrate 10, an array of electrodes 13 is provided for applying electrical current to retinal cells of an eye. Each electrode 13 is connected to a separate electrical conductor, e.g., a biocompatible metal wire 14 such as a platinum wire. As the conductors 14 extend from the electrodes 13 through the substrate 10 towards the proximal end 12 they are bunched together in helical or wavy configurations, along two spaced paths 141, 142. By using helically configured conductors or wave shaped conductors, both in the substrate 10 and elsewhere in the apparatus, upon flexing of the apparatus, the conductors can effectively expand or contract in length as necessary, avoiding damage to components of the apparatus including the conductors themselves.

The two conductor paths 141, 142 join together at the proximal end 12 of the substrate 10, adjacent an exit point of the conductors from the substrate 10. At the exit point, the conductors 14 continue along a single helical path, passing through the anchor device 2 and then extending further on through the lead 3.

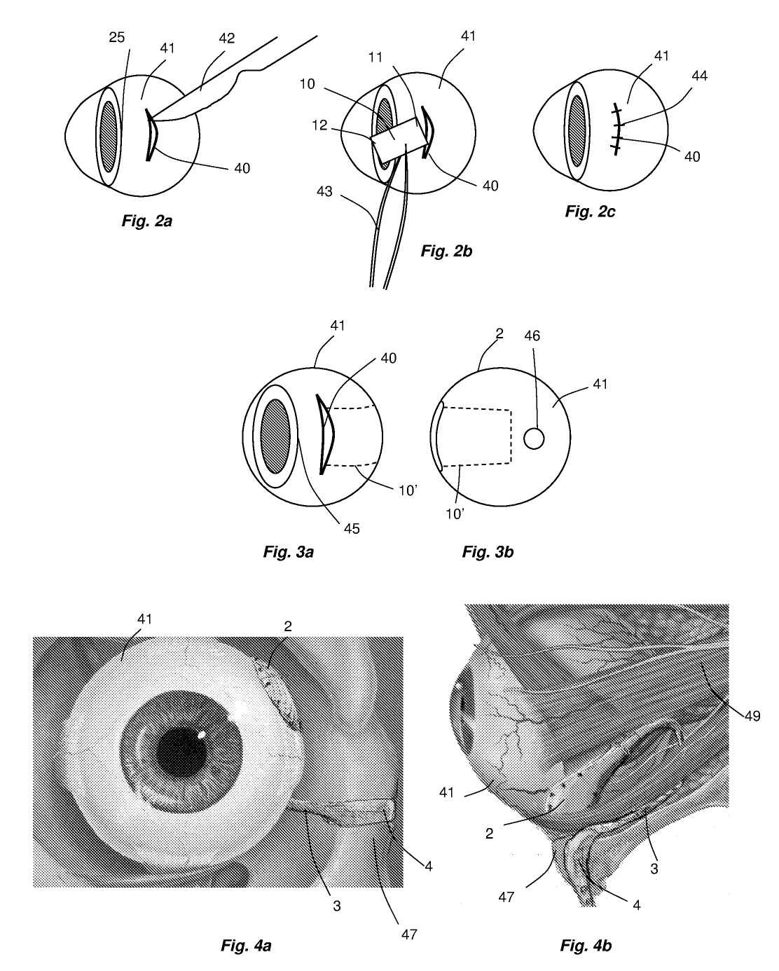

An example method of implanting the substrate 10 in an eye is now discussed with respect to FIGS. 2a to 3b. An incision 40 is made in the sclera 41 of the eye with a scalpel 42, the incision 40 being slightly wider than the width of the substrate (FIG. 2a). The distal end 11 of the substrate 10 is then pushed into the incision 40, using soft-tipped forceps 43, through the scleral layer and into a pocket between the sclera and the choroid (FIG. 2b). Once fully inserted, the opening of the incision is closed using sutures 44 (FIG. 2c). In this example, as represented in FIGS. 3a and 3b, the incision 40 is about 5 mm from the corneal limbus 45 and the substrate 10, when fully implanted, locates entirely between the sclera and choroid layers of the eye. The electrodes 13 locate adjacent the active cells of the eye's retina, about 2 mm to 4 mm, e.g. 3 mm to one side of the optic disc 46 (in FIGS. 3a and 3b, the location of the substrate 10 under the sclera is indicated by dotted lines 10′ and, for simplicity, neither the anchor device 2 nor the lead 3 is represented in FIGS. 2a to 3b).

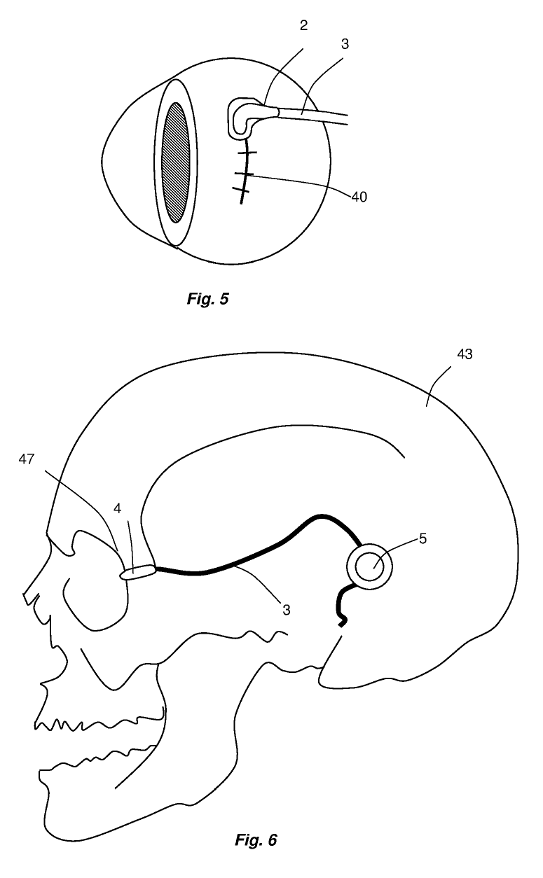

The anchor device 2 is used to stabilise the conductors 14/lead 3 at the exit point of the eye, prior to routing of the lead 3 towards a communications interface. The anchor device 2 also serves to channel the conductors 14/lead 3 in an appropriate direction away from the eye, towards the communications interface or other electronics. FIGS. 4a and 4b show front and top views, respectively, of an eye socket region showing possible location positions for the anchor device 2 in relation to the eye, and routing of the lead 3 from the eye, past the extraocular muscles 49 and around the orbital bone 47 at the lateral orbital margin. The anchor device 2, and indeed the incision 40 where it is located, are strategically positioned on the sclera 41 to avoid interfering with the extraocular muscles 49. Particularly, the incision 40 in this embodiment is positioned behind the connection between the eye and the lateral rectus muscle, and the anchor device 2 is configured to direct the lead 3 rearward, over the top of the lateral rectus muscle and between the lateral rectus muscle and the superior rectus muscle.

Referring again to FIG. 1, the anchor device 2 includes a fixation portion 21 that is configured to be secured to the sclera of the eye, and a conductor receiving portion 22 having a channel 23 through which the lead 3, including the conductors 14, extends. In this embodiment, the fixation portion 21 and receiving portion 22 are both formed at least in part of silicone, although other flexible polymers or indeed other types of materials may be used.

The fixation portion 21 includes a relatively flat piece of silicone in which a layer of polyethylene terephthalate (PET) mesh 24 is embedded, increasing the rigidity and strength of the fixation portion 21. Accordingly, while silicone covering the mesh 24 provides the fixation portion 21 with a relatively conformable surface suitable for engagement with the eye, the size and shape of the fixation portion 21 remains substantially fixed by the mesh 24. Thus, the fixation portion 21 provides a firm, relatively flat, platform for engaging and securing the anchor device 2 to the outer surface of the eye.

The conductor receiving portion 22 includes no reinforcing mesh layer in this embodiment and is therefore relatively flexible in comparison to the fixation portion 21. The receiving portion 22 maintains a gap between the channel 23 and the fixation portion 21, and thus provides a relatively flexible transition region between the channel 23, including the lead and conductors 14, and the fixation portion 21, allowing a controlled degree of movement therebetween. The movement can ensure that, while the anchor device 2 provides a secure path for the conductors 14 to exit the incision 40 in the eye, the conductors 14 may still flex, e.g. during rotation of the eye, reducing the likelihood of damage to the eye at the eye exit point, or possible breakage occurring to the conductors 14.

In this embodiment, the conductor receiving portion 22 and the channel 23 follow a bent, substantially right-angled, path between a first end 201 of the anchor device 2 adjacent the substrate 10 and a second end 202 of the anchor device 2 where the lead 3 extends from the anchor device 2 towards the communications interface or other electronics. The bend directs the lead 3 away from the incision 40 in the eye and towards the communications interface or other electronics, as can be seen in FIG. 5.

The lead 3 includes silicone cladding that surrounds the helically arranged conductors 14. Referring to FIG. 1, at approximately 3 to 4 cm along the lead from the substrate, the lead 3 is provided with a reinforcement device, also known as a grommet 4, that both directs the conductors 14 around the orbital bone 47 of the eye socket, as shown in FIGS. 4a and 4b, and provides protection for the conductors 14 against high stresses at this region. After extending around the orbital bone 47, as shown in FIG. 6, the lead 3 extends along the side of the patients skull 43 to the communications interface (which is a plug pedestal 5 in this embodiment) or other electronics. The communications interface is adapted to receive a stimulation signal for transmitting to the electrodes and applying to retinal cells to produce a visual percept. The stimulation signal is based on images captured by an image capture device, such as a video camera. The image capture device is connected to a processor and the processor directs production of the stimulation signal in accordance with the images.

Eye tracking functionality is provided in the apparatus. Tracking of the position and/or movement of the eye enables spatially accurate information to be provided to the patient. When a direction of eye gaze of the patient changes, tracking of the eye enables an analogous change in direction of the visual percept to be elicited.

For the purposes of eye tracking, a first inductor, specifically a first coil 143, also referred to hereinafter as an “internal coil” 143, is provided in the substrate 10, adjacent the proximal end 12 of the substrate (see FIG. 1). The coil 143 is formed from conductive wire. In this embodiment, the internal coil 143 is encapsulated within silicone forming the substrate 10. In alternative embodiments, the coil can be located on a surface of the substrate, and/or at different position of the substrate, e.g. at a central or proximal region of the substrate, or elsewhere in the apparatus. The internal coil 143 is electrically connected to the communications interface 5 via one or more conductors extending through the lead 3, allowing currents induced in the coil 143 to be sensed and monitored at an external location. In an alternative embodiment, the electrical connection allows current to be supplied to the coil 143 to create magnetic field about the coil 143.

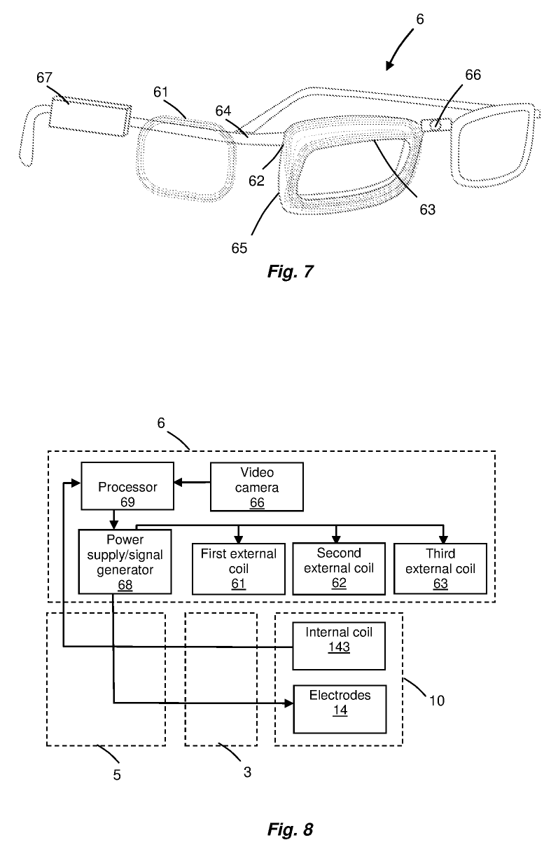

Referring to FIG. 7, the apparatus further includes eyewear in the form of spectacle frames 6 which are worn by the patient. A plurality of second inductors, specifically second coils, which can be considered “external coils” at least in this embodiment, are provided on the frames 6. In particular, first, second and third external coils 61, 62 and 63, are provided at locations of the frames 6 adjacent the eye in which the electrical stimulation is carried out. The first external coil 61 is provided on an arm 64 of the frames 6, the second external coil 62 is provided on a lens rim 65 of the frames 6, and the third external coil 63 is provided on the same lens rim 65, but at a different angle from the second external coil 62. The arrangement is such that the coil axes of the three coils 61, 62, 63 lie orthogonally or at a substantial angle to each other. An image capture device in the form of a video camera 66 is located on the nose bridge of the spectacles 6. Further, a housing 67 is provided on the arm 64 that contains a power supply/signal generator 68 and a processor 69.

Referring to FIG. 8, which shows a schematic drawing of various components of the apparatus of FIGS. 1 and 7, the video camera 66 is adapted to transmit video signals to a processor 69, which processor 69 causes the signal generator 68 to produce a stimulation signal based on the video signals for transmission to the electrodes 14 via the communications interface 5 and lead 3. Further, alternating current signals are supplied from the power supply/signal generator 68 to each of the first, second and third external coils 61, 62, 63 at different respective frequencies, causing magnetic fields with different frequencies to be produced around the three coils 61, 62, 63. When the frames 6 are worn, the internal coil 143 sits within the different magnetic fields, resulting in the induction of an electrical current signal within the internal coil 143. During movement of the eye, the internal coil 143 moves within the different magnetic fields, causing changes in the induced electrical current signal within the internal coil 143. The induced signal is transmitted to the processor 69 for demodulation on a frequency-specific basis, allowing the processor to determine the degree of movement of the eye along three orthogonal axes based on changes in the induced signal, and thus to determine changes in direction of eye gaze.

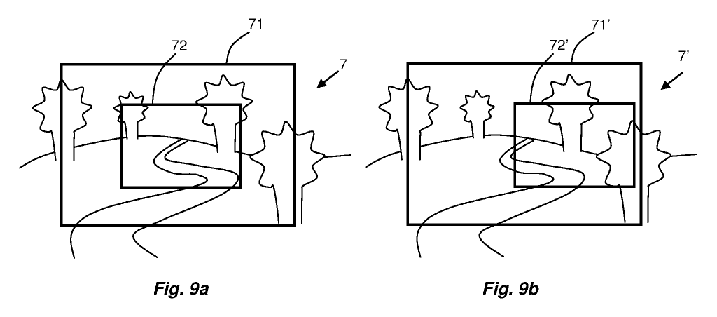

Based on changes in the determined direction of eye gaze, the processor 69 is adapted to modify the stimulation signal such as to provide a visual percept of selecting or scanning over a portion of a visual scene. To facilitate this operation, in this embodiment, the video camera 66 has a relatively wide field of view and only a section of each of the video signal images it receives is used to generate the stimulation signal. Thus, if, for example, the patient's eye gaze is determined to be in a left of centre direction, the processor is configured to adjust the stimulation signal such that the signal is based on a left of centre portion of one or more of the video signal images. Similarly, if, for example, the patient's eye gaze is determined to be in a bottom of centre direction, the control module is configured to adjust the stimulation signal such that the stimulation signal is based on a bottom of centre portion of one or more of the video signal images. To aid understanding of this technique, FIGS. 9a and 9b are provided, which illustrate how different portions of images captured by the video camera can be selected in consideration of determined movement of the eye. Referring to FIG. 9a, a portion of a visual scene 7, located in front of the patient, captured as first video image at a first point in time, is represented by rectangle 71. In FIG. 9b, a portion of the visual scene 7, captured as a second video image at a second point in time, is represented by rectangle 71′. Between the capture of the first and second video images 71, 71′, the patient has maintained his/her head, and thus the video camera, in substantially the same forward-facing position, and accordingly the positioning of the first and second video images 71, 71′ relative to the visual scene 7 is substantially identical. At the first point in time, the patient's direction of eye gaze is determined through tracking to be in a substantially central direction. Accordingly, the processor is configured such that the stimulation signal, at substantially the first point in time, is based on a central portion 72 of the first video image 71. At the second point in time the patient's direction of eye gaze is determined by the processor to have moved to a right of centre direction. Accordingly, the processor is configured such that the stimulation signal, at substantially the second point in time, is generated based on a right of centre portion 72′ of the second video image 71′. Thus, between the stimulation based on the first and second video images, the visual percept experienced by the patient gives the patient the impression of scanning to the right of a visual scene.

The same technique can also give the patient the impression of maintaining fixation on the same portion of a visual scene while their head, and thus the video camera, scans across the scene. This functionality is akin to the vestibulo-ocular reflex, providing for stabilization of images during head movement, experienced by healthy-sighted individuals.

In alternative embodiments, in addition to or as an alternative to selecting different portions of the video signals to gives the patient the impression of scanning across a visual scene, or maintaining fixation on the same portion of a moving visual scene, the video camera is moved based on the determined direction of eye gaze. The video camera can be pivotally mounted on the eyewear, for example, and one or more motors or other actuators can be used to cause rotation of the video camera, e.g. around one, two or three orthogonal axes.

Using the eye movement monitoring techniques described herein, adaptation of the stimulation signal and/or movement of the image capture device may also be used to counteract effects of involuntary eye movement such as nystagmus. Since eye movement can change the perceived position of a visual percept, and this can be particularly problematic in the case of involuntary eye movement, adaptation of the stimulation signal and/or directing of movement of the image capture device may be at least partially based on determined involuntary eye movement.

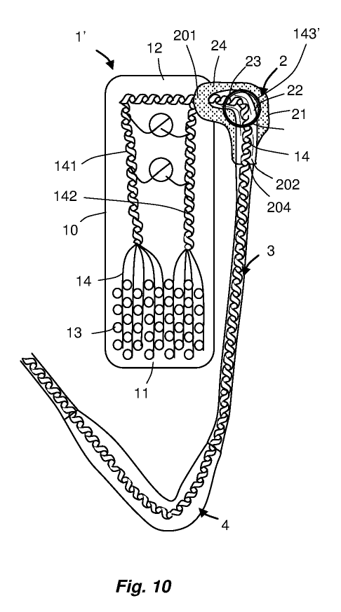

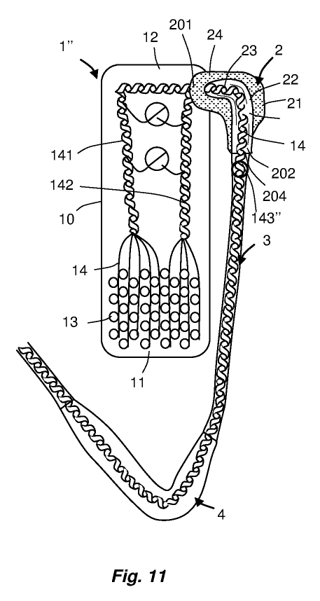

In the implantable device 1 discussed above with reference to FIG. 1, for example, the internal coil 143 is located in the substrate 10. However, in alternative embodiments, an internal coil may be located elsewhere in the implantable device 1. For example, as illustrated in FIG. 10, an internal coil 143′ can be located in the anchor device 2 of the implantable device 1′. As another example, as illustrated in FIG. 11, an internal coil 143″ can be located in the lead 3 of the implantable device 1″. By providing the internal coil 143′ in a portion of the implantable device that is not positioned within the eye, but which is substantially fixed in position in relation to the eye, the device may be less invasive. For example, to the extent that the substrate does not include the coil, the substrate may take a more streamlined configuration, making it easier to insert into the eye and/or ensuring it causes less damage to the eye. Moreover, locating the internal coil 143′, 143″ in the anchor device 2 or lead 3 may move the internal coil 143′, 143″ closer to the external coils 61, 62, 63, thus providing for stronger inductive coupling.

While first and second inductors in the form of first and second coils are employed in embodiments described above, the first and second inductors may take different forms. For example, one or both of the first and second inductors may comprise only a single loop or a partial loop of wire.

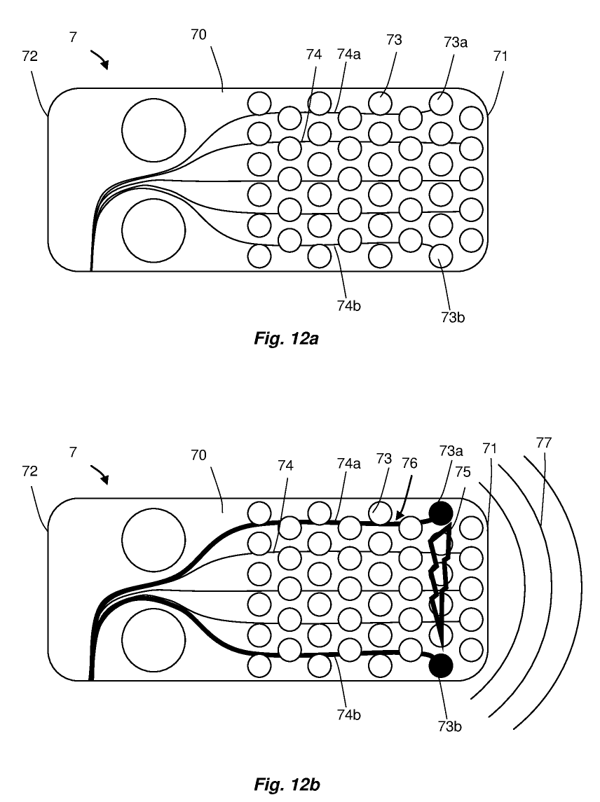

Visual prosthesis apparatus according to an embodiment of the present disclosure in which the first inductor comprises a partial loop is illustrated in FIGS. 12a and 12b. An implantable device 7 is provided having a flexible substrate 70 with a distal end 71 and a proximal end 72. The substrate 70, when viewed from above, is substantially rectangular, with curved corners to minimise surgical trauma, its longitudinal direction extending between the distal and proximal ends 71, 72. Adjacent the distal end 71 of the substrate 70, an array of electrodes 73 is provided for applying electrical current to retinal cells of an eye. Each electrode 73 is connected to, and addressable using, a separate electrical conductor 74, e.g., a biocompatible metal wire such as a platinum wire (only some of the conductors 74 are illustrated in FIGS. 12a and 12b). As the conductors 74 extend from the electrodes 73 through the substrate 70 towards the proximal end 72 they bunch together before travelling through a lead (now shown).

In this embodiment, as shown in FIG. 12b, a partial loop is provided by a first one of the conductors 74a that connects to a first one of the electrodes 73a, and a second one of the conductors 74b that connects to a second one of the electrodes 73b. The first and second electrodes 73a, 73b are each provided at opposite lateral sides of the substrate 70 towards the distal end 71. Similarly, the second conductors 74a, 74b extend along opposite lateral sides of the substrate 70.

Electrical current can be applied between the first and second electrodes 73a, 73b using the first and second conductors 74a, 74b, whereupon the electrical signal travels through eye tissue located between the first and second electrodes to complete a conductive loop 76. The application of an electrical current through the first and second conductors 74a, 74b and the first and second electrodes 73a, 73b is represented in FIG. 12a using dark black lines/circles. The transfer of the electrical current through the eye tissue to complete the loop 76 is represented by symbol 75.

The general function of the conductive loop 76 created in this embodiment can be similar to the first coil 143 described above with reference to FIGS. 1 to 9. Alternating current supplied to the loop can cause an alternating magnetic field to be generated, as indicated by lines 77 in FIG. 12b. The changes in electrical current induced in one or more second inductors that can be located in the magnetic field, e.g., arranged as shown in FIG. 7 or otherwise, can be monitored to identify relative movement of the loop and therefore the implantable device 7 and the eye.

The current supplied to the loop 76 for the purpose of eye tracking can be the same current that is delivered to the array of electrodes 73 for the purposes of providing a visual percept to the patient. Thus, the supply of the current can have a dual purpose: the provision of a visual percept through electrical stimulation of the eye, and the production of an electrically induced current in an inductor external to the eye for the purpose of monitoring eye movement. By combining these functions, a single current source only may be required.

In alternative embodiments, the conductive loop 76 may be employed as an inductive receiver rather than transmitter. For example, current may be applied to the one or more second inductors, rather than the conductive loop 76, and an electrical signal may be received in the conductive loop 76 as a result of induction.

As discussed above with reference to FIG. 7, for example, a plurality of external coils 61, 62, 63 may be provided that lie orthogonally to each other. This allows a determination of a degree of movement of the eye along three orthogonal axes to be made. Nevertheless, external coils or other types of external inductors may be provided that are not orthogonal to each other, while still allowing for detection of movement along different axes.

In one embodiment, one or more groups of external coils are employed that have substantially the same orientation and are located in substantially the same plane as each other, but which are displaced from each other (e.g. vertically displaced, horizontally displaced or otherwise). Only one group of external coils may be provided, or multiple groups of external coils may be provided. Where multiple groups of external coils are provided, each group may be located on a plane that is orthogonal to each other, e.g., similar to the arrangement shown in FIG. 7, or otherwise. Each group of external coils may be used in a differential configuration.

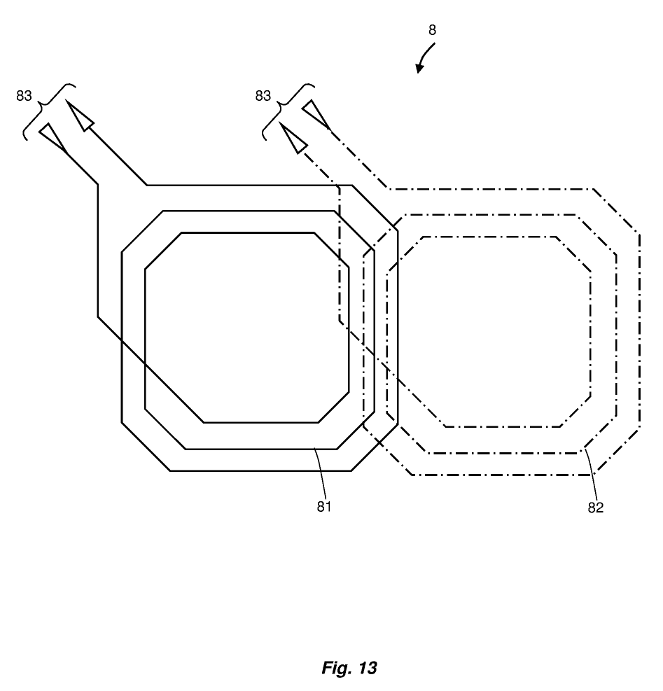

An example of a pair of coils 8 in differential configuration is illustrated schematically in FIG. 13. The pair of coils 8 comprises two external coils 81, 82. The external coils 81, 82 have substantially the same orientation and are located in substantially the same plane, but are laterally displaced from each other. Moreover, the external coils 81, 82 have opposing directions of coil winding, the directions of coil winding by indicated by arrows 83. As shown in FIG. 13, one of the external coils 81 has a clockwise winding and the other of the external coils 82 has an anticlockwise winding. The different directions of coil winding place the external coils 81, 82 in an inversely polarised relationship such that signals from the coils 81, 82 received by a processor are inverted relative to each other. An alternative approach to achieving inversion is to connect the coils 81, 82 to the processor in an inverted manner or configuring the processor to invert a signal received from one of the coils but not a signal received from the other of the coils.

Using the pair of coils 8 in differential configuration, a comparison or computation may be carried out based on differences in the induced current at each external coil 81, 82, as the external coils 81, 82 move relative to an inductor, e.g. an internal coil or loop, in the implantable device. Signals from the two coils may be summed together. The use of the pair of coils 8 in differential configuration may therefore increase the accuracy or resolution of the eye movement detection by the apparatus.

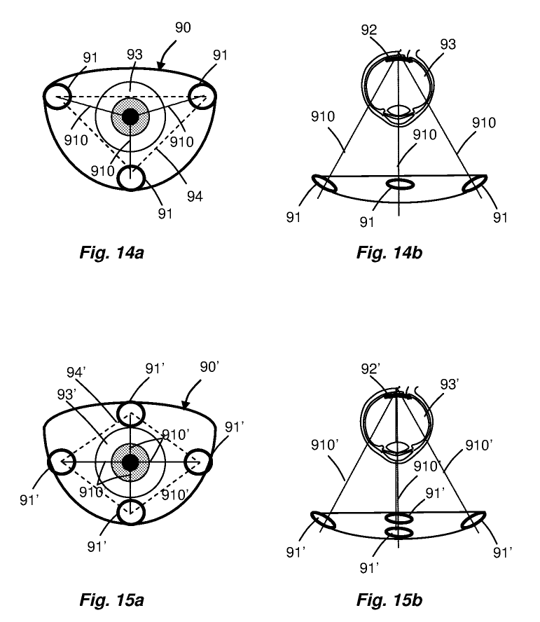

In the embodiment discussed with reference to FIG. 7, the second inductors 61, 62, 63 are positioned substantially orthogonally to each other. In some alternative embodiments, as now discussed with reference to FIGS. 14a to 15b, the second inductors are oriented at other angles, e.g. angles dependent on their positioning relative to the first inductor.

In the embodiments illustrated in FIGS. 14a to 15b, second inductors 91, 91′ are provided that are angled so that the axis 910, 910′ (e.g. coil axis) of each second inductor 91, 91′ points substantially towards the first inductor 92 located in or on the eye 93, 93′.

In each embodiment, the second inductors are provided at different locations of a lens frame 90, 90′. In the embodiment illustrated in FIGS. 14a and 14b, three second inductors 91 are provided, the second inductors 91 being positioned in a triangular arrangement where each of the second inductors 91 is located at the apex of a notional triangle 94. In the embodiment illustrated in FIGS. 15a and 15b, four second inductors 91′ are provided, the second inductors 91′ being positioned in a square, rectangular, rhombic or trapezoidal arrangement where each of the second inductors 91′ is located at the apex of a notional square, rectangle, rhombus or trapezoid 94′.

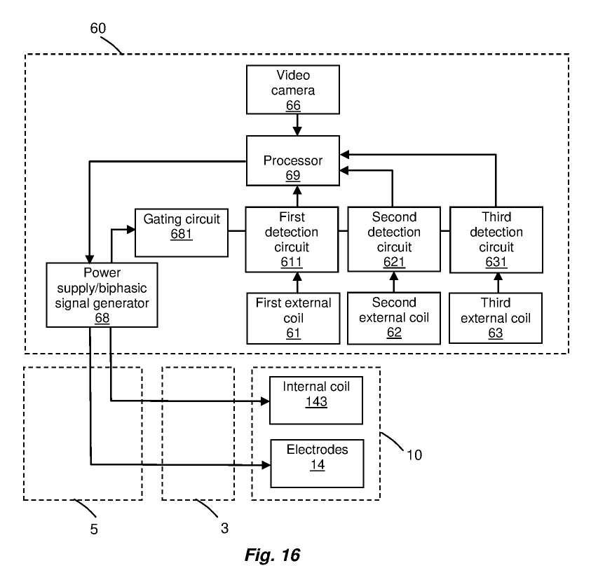

In certain preceding embodiments, e.g. as illustrated in FIG. 8, electrical current is applied by a power supply/signal generator 68 to the second inductors (e.g. external coils 61, 62, 63), in order to induce a current signal in the first inductor (e.g. internal coil 143) that is sensed to track eye movement. As described with reference to the embodiment of FIGS. 12a and 12b, for example, an alternative approach is to apply electrical current to the first inductor in order to induce current signals in the second inductors that are sensed to track eye movement. A further example of this approach is provided in the embodiment now discussed with reference to FIG. 16.

FIG. 16 shows a schematic drawing of various components of the apparatus, including eye glass frames 60 housing or supporting a video camera 66 that is adapted to transmit video signals to a processor 69, which processor 69 causes a signal generator 68 to produce a stimulation signal based on the video signals for transmission to electrodes 14 of a substrate implanted in an eye, via the communications interface 5 and lead 3. In this embodiment, alternating current signals are concurrently supplied from the power supply/signal generator 68 to the internal coil 143, rather than the first, second and third external coils 61, 62, 63. This causes a magnetic field to be produced around the internal coil 143, resulting in induction of an electrical current signal within the external coils 61, 62, 63.

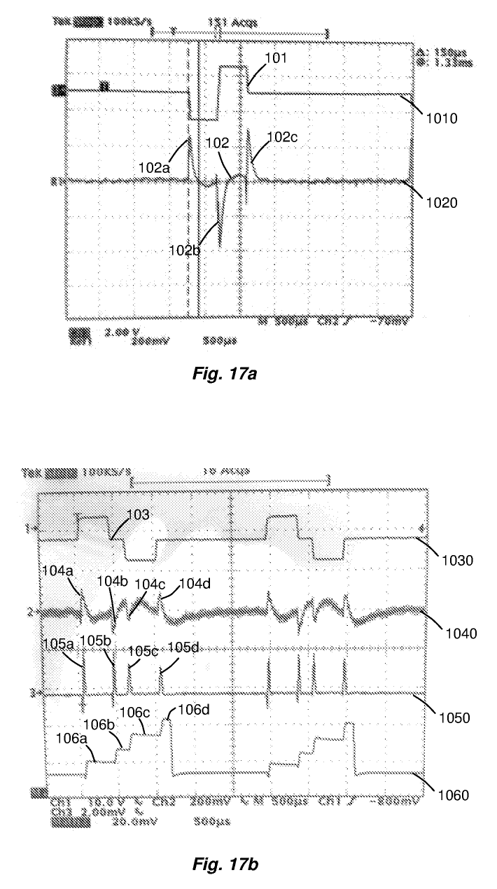

In this embodiment, the electrical current is applied to the internal coil 143 as a series of biphasic pulses, the biphasic pulses ensuring that there is no net charge transfer to the eye 10. A graph 100 indicating the voltage profile of a biphasic pulse 101 is illustrated in FIG. 17a (see top line 1010). Each biphasic pulse induces a current flow in the external coils 61, 62, 63 that has a specific voltage profile 102, an example of which is illustrated in the bottom line 1020 of FIG. 17a. The induced current has a voltage profile 102 that includes a plurality of abrupt changes 102a-c of different magnitudes, each change caused by a change in voltage direction of the biphasic pulse 101.

During movement of the eye, the external coils 61, 62, 63 move relative to the magnetic field around the internal coil 143, causing changes in the induced electrical current signal within the external coils 61, 62, 63, the changes being reflected by variations in the respective voltage profiles 102.

Referring again to FIG. 16, in this embodiment a detector module is provided comprising first, second and third detection circuits 611, 621, 631, each associated with a respective one of the first, second and third external coils 61, 62, 63. The detection circuits 611, 621, 631 detect each spike 102a-c in the induced voltage profile 102 at the respective external coil 61, 62, 63 and provide a signal output to the processor 69.

The processing by the detector module is illustrated in more detail with reference to FIG. 17b. Like FIG. 17a, FIG. 17b includes two signal lines 1030, 1040 that illustrate the raw biphasic pulses 103 applied to the first inductor 143, and the signal 104, including spikes 104a-d, that is induced in one of the second inductors 61, 62, 63, respectively. FIG. 17b also includes a signal line 1050 illustrating a signal output from processing of the induced signal 1040 by the detector module. The processing converts each negative spike 104b, 104c into a positive spike 105b, 105c, normalises the signal levels, and also filters out signal noise. Noise is filtered through use of a gating circuit 681 that ensures that the induced current is examined at specific times only, i.e. at the known instances where changes in slope polarity at the second inductor will occur. By setting a zero baseline at the instant before or between the changes in slope polarity, a processed signal 1050 for a specific time period can be output by the detector module in which background noise is substantially eliminated.

Referring to the fourth signal line 1060 of FIG. 17b, the signal 1050 output from the detector circuit is integrated over a time period during which a complete biphasic pulse is delivered to the first inductor 143. The detector module summates the integrated signal over the time period to arrive at a summated signal value (corresponding to the top level 106d of line 1060, for example).

Thus, when electrical current is supplied to the first inductor 143 implanted in the eye, inducing electrical current at multiple second inductors 61, 62, 63 external to the eye, a position of the eye may be determined by comparing a signal value such as a summated signal value 106d at each of the second inductors at an instant in time, or by comparing average signal values such as average summated values 106d at each second inductor over a time period. The comparing of signal values 106d can comprise determining a ratio of the values 106d between each of the second inductors 61, 62, 63. The comparing of the values 106d can be used to determine a position of the eye and/or movement of the eye. The ratio may be referenced to a look-up table, the look-up table providing pre-determined eye position data for different ratios.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. Visual prosthesis apparatus comprising:
an implantable device comprising a substrate and a plurality of electrodes located in or on the substrate, the substrate adapted to be implanted at least partially in an eye of a patient;
a first inductor comprised in the implantable device; and
a second inductor adapted to locate externally to the eye and inductively couple with the first inductor; and
a processor adapted to determine a direction of movement of the eye based on changes in electrical current induced in one of the first and second inductors due to relative movement of the first and second inductors.

2. The apparatus of claim 1, wherein at least one of the first and second inductors is a coil.

3. (canceled)

4. The apparatus of claim 1, wherein the first inductor is a first coil and the second inductor is a second coil.

5. The apparatus of claim 1, wherein the first inductor is a loop or partial loop.

6. The apparatus of claim 1, wherein the first inductor is located in or on the substrate.

7. The apparatus of claim 1, wherein the electrodes are located at or adjacent a distal end of the substrate and the first inductor is located at or adjacent a proximal end of the substrate.

8. The apparatus of claim 1, wherein the first inductor is a partial loop comprising a first conductor connected to a first electrode of the plurality of electrodes and a second conductor connected to a second electrode of the plurality of electrodes, wherein the first and second electrodes are adapted to electrically connect to each other through tissue of the eye to provide a complete loop.

9. The apparatus of claim 8, wherein the processor is adapted to make the determination of the direction of movement of the eye based on electrical current induced in the second inductor as a result of a stimulation signal supplied to the plurality of electrodes for the purpose of stimulating the eye to provide a visual percept to the patient.

10. The apparatus of claim 8 comprising a single current source only that supplies current to the plurality of electrodes.

11-12. (canceled)

13. The apparatus of claim 1, wherein the implantable device comprises a lead adapted to extend from the substrate to a region external to the eye to enable communication with the electrodes, wherein the first inductor is located in or on the lead.

14-16. (canceled)

17. The apparatus of claim 1, wherein the implantable device comprises an anchor device adapted to fix to the outer surface of the sclera of the eye and adapted to support one or more conductors extending out of an opening in the outer surface of the eye, wherein the first inductor is located in or on the anchor device.

18. The apparatus of claim 1, comprising eyewear, wherein the second inductor is comprised in the eyewear.

19. (canceled)

20. The apparatus of claim 1, wherein the second inductor comprises a plurality of second inductors.

21. The apparatus of claim 20, wherein the plurality of second inductors are configured according to one of the following:
two or more of the second inductors have orthogonal coil axes;
two or more of the second inductors have coil axes that extend from different locations to the centre of the eye; and
the plurality of second inductors are arranged at corners of a notional triangle, square, rectangle, rhombus or trapezoid.

22-24. (canceled)

25. The apparatus of claim 20, wherein two or more of the second inductors are configured in a group and wherein the two or more second inductors configured in a group have substantially the same orientation and are located in substantially the same plane as each other.

26. The apparatus of claim 20, wherein:
two or more of the second inductors are configured in a group and one of the following applies:
the two or more second inductors configured in a group comprise at least two second coils and the two second coils have different coil winding directions from each other;
the two or more second inductors configured in a group are connected to the processor with an inverted relationship;
the processor is adapted to invert one or more signals received from the two or more second inductors configured in a group.

27. (canceled)

28. The apparatus of claim 1, comprising an image capture device adapted to locate externally to the eye and capture one or more images, wherein the processor is adapted to provide a stimulation signal to the plurality of electrodes based on the one or more captured images.

29. The apparatus of claim 28, wherein the plurality of electrodes provide part of the first inductor and the stimulation signal induces the electrical current in the second inductor that is used by the processor to make the determination about the direction of movement of the eye.

30. The apparatus of claim 28, wherein the processor adapts the stimulation signal based on the determined direction of movement of the eye.

31-35. (canceled)

36. An implantable device comprising:
a substrate and a plurality of electrodes located in or on the substrate, the substrate adapted to be implanted at least partially in an eye of a patient;
a first inductor comprised in the implantable device; the first inductor adapted to inductively couple with a second inductor located externally to the eye to enable determination a direction of movement of the eye based on changes in electrical current induced in one of the first and second inductors due to relative movement of the first and second inductors.
